(19) United States
(12) Patent Application Publication
(10) Pub. No.: US 2002/0106678 A1
(43) Pub. Date: Aug. 8, 2002

(54) cDNA clones encoding human G protein gamma subunits

(76) Inventors: Janet D. Robishaw, Catawissa, PA (US); Charles A. Kunsch, Norcross, GA (US)

Correspondence Address: HUMAN GENOME SCIENCES INC, 9410 KEY WEST AVENUE, ROCKVILLE, MD, 20850

(21) Appl. No.: 09/982,809

(22) Filed: Oct. 22, 2001

Related U.S. Application Data
(63) Continuation of application No. 08/952,772, filed on Jun. 30, 1998, now abandoned.

(51) Int. Cl. C12Q 1/68; G01N 33/53; G01N 33/567; C07K 14/705

(52) U.S. Cl. 435/6; 435/7.1; 530/350; 435/69.1; 435/325; 435/320.1

(57) ABSTRACT

Nucleic acid molecules encoding human γ₂, γ₃, γ₄, γ₅, γ₇, γ₁₀ and γ₁₁ subunits are provided. Subunit polypeptides are also provided. In addition, method of detecting mutated forms of human γ subunit and altered levels of human γ subunit are provided. Methods of identifying antagonists and agonists of the interaction of a βγ ligand with its receptor are also provided.

### BACKGROUND OF THE INVENTION

Intracellular transmission of extracellular signals is most commonly mediated by a family of guanine nucleotide-binding proteins, referred to as G proteins, that couple with various receptors and effectors to produce appropriate cellular responses. G protein-coupled receptors transduce a wide variety of signals ranging from hormones, neurotransmitters and chemoattractants to sensory stimuli such as light, odor and taste. Kunapuli et al. J. Biol. Chem. (1994) 269(14):10209-10212. The G proteins are heterotrimers, composed of α, β and γ subunits. In response to binding of the appropriate ligand, the receptor stimulates the exchange of bound GDP for GTP on the α subunit, resulting in the dissociation of the α subunit from the β and γ subunits. The GTP-bound α subunit has been shown to directly regulate the activity of downstream effectors. Gilman, A. G. Ann. Rev. Biochem. (1987) 56:615-649; Simon et al. Science (1991) 252:802-808; Birnbaumer, L. Cell (1992) 71:1069-1072. Gilman demonstrated that after dissociating from the GTP-bound α subunit, the βγ subunit exists as a tightly-associated complex in vivo. This complex has been found to regulate the activity of a specific subset of downstream effectors, including adenylyl cyclase subtypes II and IV, phospholipase A2, phospholipase C subtypes β1,2,3, and K⁺ and Ca²⁺ channels. Tang, W J. and Gilman, A. G. Science (1991) 254:1500-1503; Wickman et al. Nature (1994) 368:254-257; Clapham, D. E. and Neer, E. J. Nature (1993) 365:403-406. Thus, the G protein α and βγ subunits produce bifurcating signals that regulate the function of these effectors. Moreover, the βγ subunits can directly bind to a receptor (Phillips, W. J. and Cerione, R. A. J. Biol. Chem. (1992) 24:17032-17039) and can increase agonist-dependent phosphorylation and desensitization by directly interacting and recruiting the β-adrenergic (β-ARK) kinases to the membrane. Haga, K. and Haga, T. J. Biol. Chem. (1992) 267:2222-2227, Pitcher et al. Science (1992) 257:1264-1267. Thus, the βγ subunits are important in both the regulation of these effectors and receptor recognition.

Both the G protein β and γ subunits belong to large multigene families. Complete cDNAs encoding five distinct mammalian β subunits (β₁-β₅) have been identified thus far. Watson et al. J. Biol. Chem. (1994) 269:22150-22156. A rat heart cDNA recently identified may encode a sixth β subunit, which is 96% identical to the human β₃ subunit. Ray, K. and Robishaw, J. D. Gene (1994) 149:337-340. At the amino acid level, the β subunits are highly conserved.

In contrast, the γ subunits are much more divergent. Thus, it is believed that the γ subunit determines the functional specificity of the βγ subunit complex. Complete cDNAs representing five different γ subunits have been reported with the isolation of the γ₁ subunit from bovine retina (Hurley et al. Proc. Nat'l Acad. Sci USA (1984) 81:6948-6952), the γ₂, γ₃, and γ₇ subunits from bovine brain (Robishaw et al. J. Biol. Chem. (1989) 264:15758-15761; Gautam et al. Science (1989) 244:971-974; Gautam et al. Proc. Nat'l Acad. Sci. USA (1990) 87:7973-7977; Cali et al. J. Biol. Chem. (1992) 267:24023-24027), and the γ₅ subunit from bovine and rat liver. Fisher, K. and Aronson, N. N. Mol. Cell Biol. (1992) 12:1585-1591. The existence of a putative γ₄ subunit has also been reported with the isolation of a PCR fragment from mouse kidney and retina. Gautam et al. Proc. Nat'l Acad. Sci. USA (1990) 87:7973-7977.

In the present invention, the cDNA clones encoding human γ₂, γ₃, γ₄, γ₅, γ₇, γ₁₀ and γ₁₁ subunits have been isolated and characterized.

### SUMMARY OF THE INVENTION

In accordance with one aspect of the present invention, there are provided isolated nucleic acid molecules encoding human γ₂, γ₃, γ₄, γ₅, γ₇, γ₁₀ and γ₁₁ subunits including mRNAs, DNAs, cDNAs, genomic DNAs as well as analogs and biologically active and diagnostically or therapeutically useful fragments thereof.

In accordance with another aspect of the present invention, there are provided polypeptides which are γ₂, γ₃, γ₄, γ₅, γ₇, γ₁₀ and γ₁₁ subunits, as well as biologically active and diagnostically or therapeutically useful fragments, analogs and derivatives thereof. The polypeptides of the present invention are of human origin.

In accordance with yet a further aspect of the present invention, there are provided antibodies against such polypeptides.

In accordance with yet a further aspect of the present invention, there is provided a process for producing such polypeptides by recombinant techniques comprising culturing recombinant prokaryotic and/or eukaryotic host cells, containing a human nucleic acid sequence for either γ₂, γ₃, γ₄, γ₅, γ₇, γ₁₀ or γ₁₁ subunits, under conditions promoting expression of said polypeptides and subsequent recovery of said polypeptides.

In accordance with yet a further aspect of the present invention, there is also provided nucleic acid probes comprising nucleic acid molecules of sufficient length to specifically hybridize to human γ₂, γ₃, γ₄, γ₅, γ₇, γ₁₀ and γ₁₁ subunit sequences.

These and other aspects of the present invention should be apparent to those skilled in the art from the teachings herein.

### DETAILED DESCRIPTION OF THE INVENTION

The roles of the G protein βγ dimers in regulating the activity of a specific subset of downstream effectors including adenylyl cyclase subtypes II and IV, phospholipase A2, phospholipase C subtypes β1,2,3, and K⁺ and Ca²⁺ channels, and receptor recognition has increased the importance of identifying and characterizing these proteins. Functional specificity of these dimers is believed to be determined by the γ subunit. Striking differences between the retinal and brain βγ subunits have been reported in terms of membrane association (Lee et al. J. Biol. Chem. (1992) 267:24776-24781), interaction with G protein α subunits, receptors (Fawzi et al. J. Biol. Chem. (1991) 266:12194-12200), receptor kinases (Pitcher et al. Science (1992) 257:1264-1267), and effectors (Iniguez-Lluhi et al. J. Biol. Chem. (1992) 32:23409-23410). Since the retinal and brain βγ subunits share a common β₁ subunit, these differences appear to be due to their unique γ subunit.

In the present invention, seven human cDNA clones encoding γ subunits have been identified. Based upon identity at the amino acid level, it has been determined that four of the seven cDNA clones represent the human γ₂, γ₃, γ₅, and γ₇ subunits. The nucleotide sequences for the γ₂, γ₃, γ₅, and γ₇ subunit clones have been determined and are provided as SEQ ID NOs. 20, 21, 22 and 23, respectively. The remaining three cDNA clones do not appear to be related to any known γ subunits. The amino acid differences of these three were distributed throughout the proteins, indicating they did not arise by alternative splicing of known γ subunits. The predicted amino acid sequence of one of the three cDNA clones showed marked identity (97%) to the PCR fragment of a putative mouse γ₄ subunit (Gautam et al. Proc. Nat'l Acad. Sci. USA (1990) 87:7973-7977). Accordingly, this subunit has been designated the γ₄ subunit. The other two cDNA clones were designated γ₁₀ and γ₁₁ subunits. The complete nucleotide sequences for the γ₄, γ₁₀ and γ₁₁ subunit clones have been determined and are provided as SEQ ID NOs. 9, 10 and 11, respectively.

The cDNA clones of the γ₄, γ₁₀ and γ₁₁ subunits were deposited as ATCC Deposit No. 97140, 97138, and 97139, respectively, on May 4, 1995. A mixture of cDNA clones of the γ₂, γ₃, γ₅, and γ₇ subunits was deposited as ATCC Deposit No. 97137 on May 4, 1995. The coding region of each of these cDNAs in the mixture can be obtained by PCR amplification using the following primer pairs. For the γ₂ subunit the sense primer is 5′-CTATCCAGCACTCCGATGGC-3′ (SEQ ID NO: 12) and the antisense primer is 5′-AGACTTAAAGGATGGCACAG-3′ (SEQ ID NO: 13); for the γ₃ subunit the sense primer is 5′-TGTGGCTTCAGGATGAAAGG-3′ (SEQ ID NO: 14) and the antisense primer is 5′-GAGCTCAGAGGAGAGCACAG-3′ (SEQ ID NO: 15); for the γ₅ subunit the sense primer is 5′-GTGCACCATGTCTGGCTCCT-3′ (SEQ ID NO: 16) and the antisense primer is 5′-CACTGGATCATAAGGAGTGG-3′ (SEQ ID NO: 17); and, for the γ₇ subunit the sense primer is 5′-GATGGCAGACAATGTCAGCC-3′ (SEQ ID NO: 18) and the antisense primer is 5′-AGTTATAAAATAATACAAGG-3′ (SEQ ID NO: 19).

The cDNA for the γ₄ subunit is 689 bp in length, including 98 and 365 bp of 5′- and 3′-untranslated (UTR) sequences, respectively (SEQ ID NO: 9). The first ATG codon at position 99 has the characteristics of a translation initiator codon with the expected purines at positions −3 and +4. A second ATG codon at position 111 lacks the expected purines, making it less likely to be the initiator codon. A polyadenine sequence was observed near the 3′-end of the cDNA.

The cDNA for the γ₁₀ subunit is 1213 bp in length, including 23 and 986 bp of 5′- and 3′-UTR sequences, respectively (SEQ ID NO: 10). The long 3′-UTR possesses a poly(A) tail, a polyadenylation signal towards the 3′-end, and several A(T)_{β}A motifs implicated in mRNA stability.

The cDNA for the γ₁₁ subunit is 654 bp in length, including 106 and 326 bp of 5′- and 3′-UTR sequences, respectively (SEQ ID NO: 11). The 3′-UTR contains a polyadenylation signal and a poly(A) tail towards the 3′-end.

The present invention further relates to polypeptides having the deduced amino acid sequences SEQ ID NOs: 2, 3, 4, 5, 6, 7, and 8 or those encoded by the cDNA clones of the human γ₂, γ₃, γ₄, γ₅, γ₇, γ₁₀ or γ₁₁ subunits as well as fragments, analogs and derivatives of such polypeptides. The deduced amino acid sequence of the cDNA clone for γ₂ subunit is SEQ ID NO: 2; the deduced amino acid sequence of the cDNA clone for the γ₃ subunit is SEQ ID NO: 3; the deduced amino acid sequence of the cDNA clone for the γ₄ subunit is SEQ ID NO: 4; the deduced amino acid sequence of the cDNA clone for the γ₅ subunit is SEQ ID NO: 5; the deduced amino acid sequence of the cDNA clone for the γ₇ subunit is SEQ ID NO: 6; the deduced amino acid sequence of the cDNA clone for the γ₁₀ subunit is SEQ ID NO: 7; and the deduced amino acid sequence of the cDNA clone for the γ₁₁ subunit is SEQ ID NO: 8.

Comparison of the protein sequences predicted to be encoded by the cDNA of the γ₄, γ₁₀ and γ₁₁ subunits to the homologs of the γ₁ (SEQ ID NO: 1), γ₂, γ₃, γ₅ and γ₇ subunits revealed significant homology. For the γ₄ subunit, the homology ranged from a low of 38% for the γ₁ subunit to a high of 77% for the γ₂ subunit. For the γ₁₀ subunit, the homology ranged from a low of 35% for the γ₄ subunit to a high of 53% for the γ₂, γ₅ and γ₇ subunits. This relatively low level of homology suggests that the γ₁₀ subunit may represent a new subclass that is only distantly related to the other γ subunits. For the γ₁₁ subunit, the homology ranged from a low of 33 to 44% for the γ₂, γ₃, γ₅ and γ₇ subunits to a high of 76% for the γ₁ subunit.

Analysis of the amino acid sequence conservation suggests that the γ subunit family can be divided into four distinct subclasses, one containing γ₁ and γ₁₁ subunits, a second containing the γ₂, γ₃, γ₄ and γ₇ subunits, a third containing the γ₅ subunit, and a fourth containing the γ₁₀ subunit. These subclasses are based not only on homology, but also on functional similarities. Thus, within a subclass, members display similar post-translational modifications and similar abilities to interact with the β and α subunits of the G proteins. For example, the γ₁ and γ₁₁ subunits, which comprise one subclass, are modified by a farnesyl group, do not interact with the β₂ subunit, and do not interact with the α₀ subunit. In contrast, the γ₂, γ₃, γ₄ and γ₇ subunits, which comprise another subclass, are modified by a geranylgeranyl group, interact with the β₂ subunit, and interact at least to some extent, with the α₀ subunit.

The terms “fragment,” “derivative” and “analog” when referring to the polypeptides provided in the sequence listing, or those encoded by the deposited cDNA, means a polypeptide which retains essentially the same biological function or activity as such polypeptide. Thus, an analog includes a proprotein which can be activated by cleavage of the proprotein portion to produce an active mature polypeptide.

The polypeptide of the present invention may be a recombinant polypeptide, a natural polypeptide or a synthetic polypeptide, preferably a recombinant polypeptide.

The fragment, derivative or analog of the polypeptides provided in the sequence listing or those encoded by the deposited cDNA way be (i) one in which one or more of the amino acid residues are substituted with a conserved or non-conserved amino acid residue (preferably a conserved amino acid residue) and such substituted amino acid residue may or may not be one encoded by the genetic code; (ii) one in which one or more of the amino acid residues includes a substituent group; (iii) one in which the mature polypeptide is fused with another compound, such as a compound to increase the half-life of the polypeptide (for example, polyethylene glycol); or (iv) one in which the additional amino acids are fused to he mature polypeptide, such as a leader or secretory sequence or a sequence which is employed for purification of the mature polypeptide or a proprotein sequence. Such fragments, derivatives and analogs are deemed to be within the scope of those skilled in the art from the teachings herein.

The polypeptides and polynucleotides of the present invention are preferably provided in an isolate form, and preferably are purified to homogeneity.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

The present invention also relates to vectors which include the cDNA clones of the present invention, host cells which are genetically engineered with vectors of the invention and the production of polypeptides of the invention by recombinant techniques.

Host cells are genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the γ subunit genes. The culture conditions, such as temperature, pH and the like, are those previously used with the host cell selected for expression, and will be apparent to the ordinarily skilled artisan.

The cDNA clones of the present invention may be employed for producing polypeptides by recombinant techniques. Thus, for example, the cDNA clone may be included in any one of a variety of expression vectors for expressing a polypeptide. Such vectors include chromosomal, nonchromosomal and synthetic DNA sequences, e.g., derivatives of SV40; bacterial plasmids; phage DNA, baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host.

The appropriate clone may be inserted into the vector by a variety of procedures. In general, the cDNA sequence is inserted into an appropriate restriction endonuclease site(s) by procedures known in the art. Such procedures and others are deemed to be within the scope of those skilled in the art.

The cDNA sequence in the expression vector is operatively linked to an appropriate expression control sequence(s) (promoter) to direct mRNA synthesis. As representative examples of such promoters, there may be mentioned: LTR or SV40 promoter, the E. coli, lac or trp, the phage lambda P_{L} promoter and other promoters known to control expression of genes in prokaryotic or eukaryotic cells or their viruses. The expression vector also contains a ribosome binding site for translation initiation and a transcription terminator. The vector may also include appropriate sequences for amplifying expression.

In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The vector containing the appropriate cDNA clone as hereinabove described, as well as an appropriate promoter or control sequence, may be employed to transform an appropriate host to permit the host to express the protein.

As representative examples of appropriate hosts, there may be mentioned: bacterial cells, such as E. coli, Streptomyces, Salmonella typhimurium; fungal cells, such as yeast, insect cells such as Drosophila S2 and Spodoptera Sf9; animal cells such as CHO, COS or Bowes melanoma; adenoviruses; plant cells, etc. The selection of an appropriate host is deemed to be within the scope of those skilled in the art from the teachings herein.

More particularly, the present invention also includes recombinant constructs comprising one or more of the sequences as broadly described above. The constructs comprise a vector, such as a plasmid or viral vector, into which a sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. The following vectors are provided by way of example; Bacterial: pQE70, pQE60, pQE-9 (Qiagen), pBS, pD10, phagescript, psiX174, pbluescript SK, pbsks, pNH8A, pNH16a, pNH18A, pNH46A (Stratagene); ptrc99a, pKK223-3, pKK233-3, pDR540, pRIT5 (Pharmacia); Eukaryotic: pWLNEO, pSV2CAT, pOG44, pXTl, pSG (Stratagene) pSVK3, pBPV, pMSG, pSVL (Pharmacia). However, any other plasmid or vector may be used as long as they are replicable and viable in the host. In addition, a complete mammalian transcription unit and a selectable marker can be inserted into a prokaryotic plasmid. The resulting vector is then amplified in bacteria before being transfected into cultured mammalian cells. Examples of vectors of this type include pTK2, pHyg and pRSVneo.

Promoter regions can be selected from any desired gene using CAT (chloramphenicol acetyl transferase) vectors or other vectors with selectable markers. Two appropriate vectors are pKK232-8 and pCM7. Particular named bacterial promoters include lacI, lacZ, T3, T7, gpt, lambda P_{R}, P_{L} and trp. Eukaryotic promoters include CMV immediate early, HSV thymidine kinase, early and late SV40, LTRs from retrovirus, and mouse metallothionein-L Selection of the appropriate vector and promoter is well within the level of ordinary skill in the art.

In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be performed by calcium phosphate transfection, DEAE-dextran mediated transfection, Polybrene, protoplast fusion, liposomes, direct microinjection into the nuclei, scrape loading or electroporation.

The constructs in host cells can be used in a conventional manner to produce the gene product encoded by the cDNA clone. Alternatively, polypeptides of the invention can be synthetically produced by conventional peptide synthesizers.

Mature proteins can be expressed in mammalian cells, yeast, bacteria, or other cells under the control of appropriate promoters. Cell-free translation systems can also be employed to produce such proteins using RNAs derived from the DNA constructs of the present invention both in vitro and in vivo. Appropriate cloning and expression vectors for use with prokaryotic and eukaryotic hosts are described by Sambrook et al., “Molecular Cloning: A Laboratory Manual”, Second Edition, Cold Spring Harbor, N.Y., (1989), the disclosure of which is hereby incorporated by reference.

Transcription of DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Examples including the SV40 enhancer on the late side of the replication origin bp 100 to 270, a cytomegalovirus early promoter enhancer, the polyoma enhancer on the late side of the replication origin, and adenovirus enhancers.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), α-factor, acid phosphatase, or heat shock proteins, among others. The heterologous structural sequence is assembled in appropriate phase with translation initiation and termination sequences, and preferably, a leader sequence capable of directing secretion of translated protein into the piroplasmic space or extracellular medium. Optionally, the heterologous sequence can encode a fusion protein including an N-terminal identification peptide imparting desired characteristics, e.g., stabilization or simplified purification of expressed recombinant product.

Useful expression vectors for bacterial use are constructed by inserting a structural DNA sequence encoding a desired protein together with suitable translation initiation and termination signals in operable reading phase with a functional promoter. The vector will comprise one or more phenotypic selectable and nonselectable markers and an origin of replication to ensure maintenance of the vector and to, if desirable, provide amplification within the host. Suitable prokaryotic hosts for transformation include E. coli, Bacillus subtilis, Salmonella ryphimurium and various species within the genera Pseudomonas, Streptomyces, and Staphylococcus, although others may also be employed as a matter of choice. In addition, a complete mammalian transcription unit and a selectable marker can be inserted into a prokaryotic plasmid. The resulting vector is then amplified in bacteria before being transfected into cultured mammalian cells.

As a representative but nonlimiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM1 (Promega Biotec, Madison, Wiss., USA). These pBR322 “backbone” sections are combined with an appropriate promoter and the structural sequence to be expressed.

Following transformation of a suitable host strain and growth of the host strain to an appropriate cell density, the selected promoter is induced by appropriate means (e.g., temperature shift or chemical induction) and cells are cultured for an additional period.

Cells are typically harvested by centrifugation, disrupted by physical or chemical means, and the resulting crude extract retained for further purification.

Microbial cells employed in expression of proteins can be disrupted by any convenient method, including freeze-thaw cycling, sonication, mechanical disruption, or use of cell lysing agents, such methods are well know to those skilled in the art.

Various mammalian cell culture systems can as be employed to express recombinant protein. Examples of mammalian expression systems include COS and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice and polyadenylation sites may be used to provide the required nontranscribed genetic elements.

Larger quantities of protein can be obtained from cell lines carrying amplified copies of the gene of interest. In this method, the gene is attached to a segment of DNA that carries a selectable marker and transfected into the cells, or are cotransfected into the cells. Sublines are then selected in which the number of copies of the gene are greatly amplified. There are a wide variety of selectable markers available in the art. For example, the dhfr gene is extensively used for coamplification. After several months of growth in progressively increasing concentrations of methotrexate, cell lines can be obtained that carry up to 1000 copies of the dhfr gene.

The polypeptide can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the mature protein. Finally, high performance liquid chromatography (HPLC) can be employed for final purification steps.

The polypeptides of the present invention may be a naturally purified product, or a product of chemical synthetic procedures, or produced by recombinant techniques from a prokaryotic or eukaryotic host (for example, by bacterial yeast, higher plant, insect and mammalian cells in culture). Depending upon the host employed in a recombinant production procedure, the polypeptides of the present invention may be glycosylated or may be non-glycosylated. Polypeptides of the invention may also include an initial methionine amino acid residue.

There are marked differences in the tissue distribution of members of the γ subunit family. Some members, such as the γ₁, γ₂, γ₃ and γ₄ subunits, are restricted to only a few tissues, whereas others, such as the γ₅, γ₇, γ₁₀ and γ₁₁ subunits, are expressed in a wide variety of tissues. Cali et al. J. Biol. Chem. (1992) 267:24023-24027. Furthermore in most cell types within a tissue, only a certain subset of γ subunits is present. Peng et al. Proc. Nat'l Acad. Sci. USA (1992) 89:10882-10886; Hansen et al. J. Mol. Cell Cardiol. (1995) 27:471-484. It is believed that such differences in distribution are important in the number of combinatorial associations of the α, β, γ subunits into functionally distinct G proteins. Differences in subcellular localizations of various γ subunits have also been reported. Hansen et al. J. Cell Biol. (1994) 126:811-819.

The formation of distinct βγ dimers as the result of selective interactions between the different β and γ subunits identified thus far is believed to contribute to the specificity of G protein mediated signaling pathways. A summary of known βγ interactions are shown in Table 1.

TABLE 1
Selective Association of β and γ Subunits

| | γ₁ | γ₂ | γ₃ | γ₅ | γ₇ | γ₄ | γ₁₀ | γ₁₁ |
|---|---|---|---|---|---|---|---|---|
| β₁ | + | + | + | + | + | + | + | + |
| β₂ | − | + | + | + | + | + | + | − |
| β₃ | − | − | − | ND | ND | − | − | − |
| In this Table, + indicates the ability to form a βγ dimer; − indicates the inability to form a βγ dimer; and ND stands for not determined. As is seen in this Table, similar to the γ₂, γ₃, γ₅ and γ₇ subunits (Schmidt et al. J. Biol. Chem. (1992) 267:13807-13810; Pronin, A.N. and Gautam, N. Proc. Nat'l Acad. Sci. USA (1992) 89:6220-6224; Iniguez-Lluhi et al. J. Biol. Chem. (1992) | | | | | | | | |
| # 32:23409-23410; Ueda et al J. Biol. Chem. (1994) 269:4388-4395), the γ₄ and γ₁₀ subunits are able to interact with the β₁ and β₂ subunits but | | | | | | | | |
| # not the β₃ subunit. In contrast, the γ₁₁ subunit is more similar to the γ₁ subunit (Schmidt et al. J. Biol. Chem. (1992) 267:13807-13810; Pronin, A.N. and Gautam, N. Proc. Nat'l Acad. Sci. USA (1992) 89:6220-6224) in that they both interact with the β₁ subunit but not with the β₂ and β₃ | | | | | | | | |
| # subunits. | | | | | | | | |

G-proteins and their coupled receptors have been implicated in a wide variety of cellular signals ranging from hormones, neurotransmitters and chemoattractants to sensory stimuli such as light odor and taste. Kunapuli et al. J. Biol. Chem. (1994) 269(14):10209-10212. Because of the integral role of the γ subunit in determining the specificity of the βγ subunit of the G protein, mutations in the γ subunit may result in abnormal cellular signals thus causing an abnormal cellular response. “Subunits” include mRNAs, DNAs, cDNAs, and genomic DNAs.

Accordingly, the cDNAs of the present invention may be used as a diagnostic in the detection of mutated forms of human γ subunits. Such detection will allow a diagnosis of an abnormal cellular response resulting from the mutated γ subunit disease.

Individuals carrying mutations in the human gene encoding the γ₂, γ₃, γ₄, γ₅, γ₇, γ₁₀ or γ₁₁ subunit may be detected at the DNA level by a variety of techniques. Nucleic acids for diagnosis may be obtained from a patient's cells, such as from blood, urine, saliva, tissue biopsy and autopsy material. The genomic DNA may be used directly for detection or may be amplified enzymatically by using PCR (Saiki et al. Nature, 324:163-166 (1986)) prior to analysis. RNA or cDNA may also be used for the same purpose. As an example, PCR primers complementary to the nucleic acid encoding either the γ₂, γ₃, γ₄, γ₅, γ₇, γ₁₀ or γ₁₁ subunit can be used to identify and analyze mutations in these subunits. For example, deletions and insertions can be detected by a change in size of the amplified product in comparison to the normal genotype. Point mutations can be identified by hybridizing amplified DNA to radiolabeled γ₂, γ₃, γ₄, γ₅, γ₇, γ₁₀ or γ₁₁ subunit RNA or alternatively, radiolabeled γ₂, γ₃, γ₄, γ₅, γ₇, γ₁₀ or γ₁₁ subunit antisense DNA sequences. Perfectly matched sequences can be distinguished from mismatched duplexes by RNase A digestion or by differences in melting temperatures.

Sequence differences between the reference gene and genes having mutations may be revealed by the direct DNA sequencing method. In addition, cloned DNA segments may be employed as probes to detect specific DNA segments. The sensitivity of this method is greatly enhanced when combined with PCR. For example, a sequencing primer is used with double-stranded PCR product or a single-stranded template molecule generated by a modified PCR. The sequence determination is performed by conventional procedures with radiolabeled nucleotide or by automatic sequencing procedures with fluorescent-tags.

Genetic testing based on DNA sequence differences may be achieved by detection of alteration in electrophoretic mobility of DNA fragments in gels with or without denaturing agents. Small sequence deletions and insertions can be visualized by high resolution gel electrophoresis. DNA fragments of different sequences may be distinguished on denaturing formamide gradient gels in which the mobilities of different DNA fragments are retarded in the gel at different positions according to their specific melting or partial melting temperatures (see, e.g., Myers et al. Science, 230:1242 (1985)).

Sequence changes at specific locations may also be revealed by nuclease protection assays, such as RNase and S1 protection or the chemical cleavage method (e.g., Cotton et al. PNAS, USA, 85:4397-4401 (1985)).

Thus, the detection of a specific DNA sequence may be achieved by methods such as hybridization, RNase protection, chemical cleavage, direct DNA sequencing or the use of restriction enzymes, (e.g., Restriction Fragment Length Polymorphisms (RFLP)) and Southern blotting of genomic DNA.

In addition to more conventional gel-electrophoresis and DNA sequencing, mutations can also be detected by in situ analysis.

The present invention also relates to a diagnostic assay for detecting altered levels of γ₂, γ₃, γ₄, γ₅, γ₇, γ₁₀ or γ₁₁ subunits in various tissues since an over-expression of these subunits compared to normal control tissue samples can result in abnormal cellular signals. Assays used to detect levels of these subunits in a sample derived from a host are well-known to those of skill in the art and include radioimmunoassays, competitive-binding assays, Western Blot analysis and preferably an ELISA assay. An ELISA assay initially comprises preparing an antibody specific to the γ₂, γ₃, γ₄, γ₅, γ₇, γ₁₀ or γ₁₁ subunit, preferably a monoclonal antibody. In addition, a reporter antibody is prepared against the monoclonal antibody. To the reporter antibody is attached a detectable reagent such as radioactivity, fluorescence or in this example, a horseradish peroxidase enzyme. A sample is now removed from a host and incubated on a solid support, e.g., a polystyrene dish, that binds the proteins in the sample. Any free protein binding sites on the dish are then covered by incubating with a non-specific protein like BSA. Next, the monoclonal antibody is incubated in the dish during which time the monoclonal antibodies attach to either γ₂, γ₃, γ₄, γ₅, γ₇, γ₁₀ or γ₁₁ subunit attached to the polystyrene dish, depending upon the specificity of the antibody. All unbound monoclonal antibody is washed out with buffer. The reporter antibody linked to horseradish peroxidase is now placed in the dish resulting in binding of the reporter antibody to any monoclonal antibody bound to either γ₂, γ₃, γ₄, γ₅, γ₇, γ₁₀ or γ₁₁ subunit. Unattached reporter antibody is then washed out. Peroxidase substrates are then added to the dish and the amount of color developed in a given time period is a measurement of the amount of γ₂, γ₃, γ₄, γ₅, γ₇, γ₁₀ or γ₁₁ subunit present in a given volume of patient sample when compared against a standard curve.

A competition assay may be employed wherein antibodies specific to either the γ₂, γ₃, γ₄, γ₅, γ₇, γ₁₀ or γ₁₁ subunit is attached to a solid support and labeled γ₂, γ₃, γ₄, γ₅, γ₇, γ₁₀ or γ₁₁ subunit and a sample derived from the host a passed over the solid support and the amount of label detected attached to the solid support can be correlated to a quantity of γ₂, γ₃, γ₄, γ₅, γ₇, γ₁₀ or γ₁₁ subunit in the sample.

The sequences of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important fist step in correlating those sequences with genes associated with disease.

Briefly, sequences can be mapped to chromosomes by preparing PCR primers (preferably 15-25 bp) from the cDNA. Computer analysis of the 3′ untranslated region of the gene is used to rapidly select primers that do not span more than one exon in the genomic DNA, thus complicating the amplification process. These primers are then used for PCR screening of somatic cell hybrids containing individual human chromosomes. Only those hybrids containing the human gene corresponding to the primer will yield an amplified fragment.

PCR mapping of somatic cell hybrids is a rapid procedure for assigning a particular DNA to a particular chromosome. Using the present invention with the same oligonucleotide primers, sublocalization can be achieved with panels of fragments from specific chromosomes or pools of large genomic clones in an analogous manner. Other mapping strategies that can similarly be used to map to its chromosome include in situ hybridization, prescreening with labeled flow-sorted chromosomes and preselection by hybridization to construct chromosome specific-cDNA libraries.

Fluorescence in situ hybridization (FISH) of a cDNA clone to a metaphase chromosomal spread can be used to provide a precise chromosomal location in one step. This technique can be used with cDNA as short as 500 or 600 bases; however, clones larger than 2,000 bp have a higher likelihood of binding to a unique chromosomal location with sufficient signal intensity for simple detection. FISH requires use of the clones from which the expressed sequence tag (EST) was derived, and the longer the better. For example, 2,000 bp is good 4,000 is better, and more than 4,000 is probably not necessary to get good results a reasonable percentage of the time. For a review of this technique, see Verna et al., “Human Chromosomes: a Manual of Basic Techniques”, Pergamon Press, New York (1988).

Once a sequence has been mapped to a precise chromosomal location, the physical position of the sequence on the chromosome can be correlated with genetic map data. Such data are found, for example, in V. McKusick, “Mendelian Inheritance in Man” (available on line through Johns Hopkins University Welch Medical Library). The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

With current resolution of physical mapping and genetic mapping techniques, a cDNA precisely localized to a chromosomal region associated with the disease could be one of between 50 and 500 potential causative genes. (This assumes 1 megabase mapping resolution and one gene per 20 kb).

Comparison of affected and unaffected individuals generally involves first looking for structural alterations in the chromosomes, such as deletions or translocations that are visible from chromosome spreads or detectable using PCR base on that cDNA sequence. Ultimately, complete sequencing of genes from several individuals is required to confirm the presence of a mutation and to distinguish mutations from polymorphisms.

The polypeptides, their fragments or other derivatives, or analogs thereof, or cells expressing them can be used as an immunogen to produce antibodies thereto. These antibodies can be, for example, polyclonal or monoclonal antibodies. The present invention also includes chimeric, single chain, and humanized antibodies, as well as Fab fragments, or the product of an Fab expression library. Various procedures known in the art may be used for the production of such antibodies and fragments.

Antibodies generated against the polypeptides corresponding to a sequence of the present invention can be obtained by direct injection of the polypeptides into an animal or by administering the polypeptides to an animal, preferably a nonhuman. The antibody so obtained will then bind the polypeptides itself. In this manner, even a sequence encoding only a fragment of the polypeptides can be used to generate antibodies binding the whole native polypeptides. Such antibodies can then be used to isolate the polypeptide from tissue expressing that polypeptide.

For preparation of monoclonal antibodies, any technique which provides antibodies produced by continuous cell line cultures can be used. Examples include the hybridoma technique (Kohler and Milstein Nature (1975) 256:495-497), the trioma technique, the human B-cell hybridoma technique (Kozbor et al. Immunology Today (1983) 4:72), and the EBV-hybridoma technique to produce human monoclonal antibodies (Cole et al., 1985, in “Monoclonal Antibodies and Cancer Therapy”, Alan R. Liss, Inc., pp. 77-96).

Techniques described for the production of single chain antibodies (U.S. Pat. No. 4,946,778) can be adapted to produce single chain antibodies to immunogenic polypeptide products of this invention. Also, transgenic mice may be used to express humanized antibodies to immunogenic polypeptide products of this invention.

Fragments of the cDNA encoding either the γ₂, γ₃, γ₄, γ₅, γ₇, γ₁₀ or γ₁₁ subunit may also be used as a hybridization probe for a cDNA library to isolate the full length γ₂, γ₃, γ₄, γ₅, γ₇, γ₁₀ or γ₁₁ subunit gene and to isolate other genes which have a high sequence similarity to these genes or similar biological activity. Probes of this type generally have at least 20 bases. Preferably, however, the probes have at least 30 bases and generally do not exceed 50 bases, although they may have a greater number of bases. The probe may also be used to identify a cDNA clone corresponding to a full length transcript and a genomic clone or clones that contain the complete γ₂, γ₃, γ₄, γ₅, γ₇, γ₁₀ or γ₁₁ subunit gene including regulatory and promotor regions, exons, and introns. An example of a screen comprises isolating the coding region of the γ₂, γ₃, γ₄, γ₅, γ₇, γ₁₀ or γ₁₁ subunit gene by using the known DNA sequence to synthesize an oligonucleotide probe. Labeled oligonucleotides having a sequence complementary to that of the gene of the present invention are used to screen a library of human cDNA, genomic DNA or mRNA to determine which members of the library the probe hybridizes to.

The cDNA clones and polypeptides of the present invention may also be employed as research reagents and materials for discovery of treatments and diagnostics to human disease.

This invention provides a method for identification of the receptor for the selected βγ ligand. The gene encoding the receptor can be identified by numerous methods known to those of skill in the art, for example, ligand panning and FACS sorting (Coligan et al., “Current Protocols in Immun.”, 1(2), Chapter 5, (1991)). Preferably, expression cloning is employed wherein polyadenylated RNA is prepared from a cell responsive to the selected βγ ligand, and a cDNA library created from this RNA is divided into pools and used to transfect COS cells or other cells that are not responsive to the selected βγ ligand. Transfected cells which are grown on glass slides are exposed to labeled βγ ligand. The selected βγ ligand can be labeled by a variety of means including iodination or inclusion of a recognition site for a site-specific protein kinase. Following fixation and incubation, the slides are subjected to autoradiographic analysis. Positive pools are identified and sub pools are prepared and retransfected using an iterative sub-pooling and rescreening process, eventually yielding a single clone that encodes the putative receptor. As an alternative approach for receptor identification, labeled ligand can be photoaffinity linked with cell membrane or extract preparations that express the receptor molecule. Cross-linked material is resolved by PAGE and exposed to X-ray film. The labeled complex containing the ligand-receptor can be excised, resolved into peptide fragments, and subjected to protein microsequencing. The amino acid sequence obtained from microsequencing is used to design a set of degenerate oligonucleotide probes to screen a cDNA library to identify the gene encoding the putative receptor.

The present invention also provides a method of screening potential drugs to identify those which enhance (agonists) or block (antagonists) interaction of ligand to receptor. An agonist is a compound which increase the natural biologic function of particular ligands, while antagonists are compounds which eliminate these functions. For example, a mammalian cell or membrane preparation expressing a receptor for a particular βγ subunit is incubated with labeled ligand in the presence of a test compound. The ability of this test compound to act as an agonist enhancing the interaction or as an antagonist blocking the interaction can be measured. Potential antagonists may also be identified by competitive inhibition assays wherein a potential antagonist and a particular βγ subunit are combined with membrane bound βγ subunit receptor or recombinant βγ subunit receptor under appropriate assay conditions. Such appropriate assay conditions can be routinely determined by those of skill in the art. In these assays, the βγ subunit is labeled, preferably radiolabeled, so that the number of βγ subunits bound to the receptor can determine the effectiveness of the potential antagonist.

Potential antagonists include, but are not limited to, an antibody, or in some cases, an oligopeptide which binds to the βγ subunit. Alternatively, a potential antagonist may be a closely related protein which binds to the receptor site but is inactive thus preventing the action of the βγ subunit by occupying the receptor site. Another potential antagonist is an antisense construct prepared using antisense technology. Antisense technology can be used to control gene expression through triple-helix formation or antisense DNA or RNA, both of which methods are based on binding of a polynucleotide to DNA or RNA. For example, the 5′ coding region of the polynucleotide sequence which encodes for the mature polypeptide of the present invention is used to design an antisense RNA oligonucleotide from about 10 to about 40 base pads in length. A DNA oligonucleotide is designed to be complementary to a region of the gene involved in transcription (triple helix—see Lee et al. Nucl. Acids Res. (1979) 6:3073; Cooney et al. Science (1988) 241:456 and Dervan et al. Science (1991) 251:1360) thereby preventing transcription and production of either the γ₂, γ₃, γ₄, γ₅, γ₇, γ₁₀ or γ₁₁ subunit. The antisense RNA oligonucleotide hybridizes to the mRNA and blocks translation of the mRNA molecule in the γ₂, γ₃, γ₄, γ₅, γ₇, γ₁₀ or γ₁₁ subunit. These oligonucleotides can also be delivered to cells in vivo to inhibit production of γ₂, γ₃, γ₄, γ₅, γ₇, γ₁₀ or γ₁₁ subunits.

The following nonlimiting examples are provided to further illustrate the present invention.

### EXAMPLES

### Example 1: Isolation and Analysis of cDNA Clones Encoding G Protein γ Subunits

Several cDNA libraries from specific human tissues or cell lines were made by isolating poly(A)⁺RNA from tissues and cell lines using routine procedures. Partial nucleotide sequences of cDNA clones were obtained by using either T7 or T3 primers of pBluescript vector (Stratagene, La Jolla, Calif.). As a result of this large scale sequencing, several expressed sequence tags (ESTs) were generated. By matching the sequences of ESTs to genes of known sequences, the human G protein γ subunit family was systematically classified and categorized.

### Example 2: Northern Blot Hybridization

A Northern blot containing 2 μg of poly(A)⁺ mRNA prepared from several human tissues (Clontech, Palo Alto, Calif.) was hybridized at 42° C. in 50% formamide, 5×SSPE (20×SSPE=3M NaCl, 0.2 M Sodium phosphate, 0.02 M EDTA, pH 7.4), 0.1% polyvinylpyrrolidone, 0.1% bovine albumin serum and 2% sodium dodecyl sulfate, and 100 μg/ml sheared salmon sperm DNA. Fragments of the γ₄, γ₁₀ and γ₁₁ cDNAs were isolated by double digestion of tie corresponding cDNA clones in pbluescript vector with EcoRI aid XhoI restriction enzymes. Probes were generated from the purified fragments by random priming with the Klenow fragment of DNA polymerase-I in the presence of [32P]-dCTP (3,000 Ci/mmole, Amersham Corp., Arlington Heights, Ill.). After hybridization, high stringency washes were performed at 65° C. in 0.1×SSC (1×SSC is 0.15 M sodium chloride, 0.015 M sodium citrate), 0.1% SDS. Blots were exposed for the indicated times at −80° C. with an intensifying screen.

### Example 3. Construction of Plasmids

For transcription and translation purposes, the coding sequences of the human β₁, β₂, γ₄, γ₁₀, and γ₁₁ were subcloned into either pGEM (Promega, Madison, Wis.) or pBluescript vectors by PCR amplification of the corresponding cDNA clones using the appropriate oligonucleotide primers. The coding sequences were then completely sequences to confirm that no errors were introduced as the result of PCR amplification. For the β₁ subunit, a 1050 bp fragment of the human β₃ cDNA clone (Levine et al. Proc. Nat'l Acad. Sci. USA (1990) 87:2389-2393) was excised with ApaI and subcloned into the ApaI site of the Bluescript KS vector.

### Example 4: In Vitro Transcription and Prenylation Assays

Plasmid DNA (1 μg) was linearized and transcribed with T7 for the γ₂, γ₁₀ and γ₁₁ subunits or T3 for the γ₄ subunit RNA polymerase. Transcription was performed in accordance with the protocol provided with the RNA capping kit (Stratagene, La Jolla, Calif.). To assess translation, 4 μg of the resulting RNA was translated in a 50 μl reaction in the TNT-coupled rabbit reticulocyte lysate system (Promega, Madison, Wis.), using 20 μCI of [³⁵S] methionine (Amersham Corp., Arlington Heights, Ill.). To examine prenylation, RNA was translated in the TNA-coupled rabbit reticulocyte lysate system supplemented with cold methionine, using 50 μCi of either [³]-farnesyl pyrophosphate (FPP) or [³H]-geranylgeranyl pyrophosphate (GGPP). After translations were allowed to proceed for 2 hours at 30° C., a 10 μl aliquot of the [³⁵S]-labeled translation mixture or a 25 μl aliquot of the [³H]-labeled translation mix was dissolved in electrophoresis sample buffer and subjected to 15% SDS-PAGE as described by Laemmli, U. K. J. Biol. Chem. (1991) 266:19867-19870.

### Example 5: In Vitro Translation and Tryptic Proteolysis

βγ interaction was assessed by a tryptic proteolysis assay. Plasmid DNA (1 μg) for each of the β and γ subunits were co-transcribed and co-translated in the TNT-coupled rabbit reticulocyte lysate system (Promega, Madison, Wis.). The plasmid DNA for each of the γ subunits was linearized to limit the generation of translated products of higher molecular weight. Whereas both the γ₂ and γ₄ subunits were translated efficiently in this system, the γ₁₀ and γ₁₁ subunits were translated at significantly lower levels. To increase levels of the γ₁₀ and γ₁₁ subunits 2 μg of capped RNA were added to the co-transcribed β-γ mix. Alternatively, γ₁₀ and γ₁₁ subunits that had been translated separately were added to the co-translated β-γ mix. For tryptic digestion, 5 or 10 μl aliquots of the co-translated β-γ mix were digested by addition of 1 μl trypsin (1 μg) in a final volume of 20 μl (with 50 mM Na-HEPES, pH 8.0) After incubation for 1 hour at 30° C., the digestions were stopped by addition of Laemmli sample buffer and boiling for 3 minutes. Protected fragments of β were visualized by running samples on 15% SDS-PAGE gels. After electrophoresis, gels were fixed in 40% methanol/10% acetic acid mix, soaked in ENHANCE (DuPont-NEN, Boston, Mass.), and dried. The dried gels were exposed for 8 to 48 hours at −80° C.

What is claimed is:

1. A nucleic acid molecule encoding human γ₂, γ₃, γ₄, γ₅, γ₇, γ₁₀ or γ₁₁ subunit.

2. A nucleic acid molecule encoding human γ₄, γ₁₀ or γ₁₁ subunit.

3. A polypeptide comprising SEQ ID NO: 2, 3, 4, 5, 6, 7 or 8.

4. A polypeptide comprising SEQ ID NO: 4, 7 or 8.

5. A method of detecting mutated forms of human γ subunits in a patient comprising:
(a) obtaining a sample of cells from a patient;
(b) isolating genomic DNA from the cells; and
(c) comparing isolated genomic DNA with PCR primers complementary to nucleic acid sequences encoding either γ₂, γ₃, γ₄, γ₅, γ₇, γ₁₀ or γ₁₁ to detected mutated forms of human γ subunits,
wherein mutated form of human γ subunits have deletions or insertions in the genomic DNA.

6. A method of detecting mutated forms of human γ subunits in a patient comprising:
(a) obtaining a sample of cells from a patient;
(b) isolating genomic DNA from the cells;
(c) hybridizing genomic DNA to radiolabeled γ₂, γ₃, γ₄, γ₅, γ₇, γ₁₀ or γ₁₁ RNA or antisense DNA sequences; and
(d) distinguishing matched sequences from mismatched duplexes to detected mutated forms of human γ subunits,
wherein mutated form of human γ subunits have point mutations.

7. A method of detecting altered levels of γ₂, γ₃, γ₄, γ₅, γ₇, γ₁₀ or γ₁₁ subunits in various tissues of a host comprising:
(a) obtaining a tissue sample from a host;
(b) incubating the tissue sample on a solid support so that proteins in the tissue sample binds to the solid support;
(c) incubating antibody specific to an γ₂, γ₃, γ₄, γ₅, γ₇, γ₁₀ or γ₁₁ subunit with the solid support so that the antibody binds to any γ₂, γ₃, γ₄, γ₅, γ₇, γ₁₀ or γ₁₁ subunit bound to the solid support; and
(d) detecting any antibody bound to the solid support to determine the level of γ₂, γ₃, γ₄, γ₅, γ₇, γ₁₀ or γ₁₁ subunit in the tissue sample.

8. A method of screening test compounds to identify agonists and antagonists of the interaction of a βγ ligand to its receptor comprising:
(a) incubating a mammalian cell or membrane preparation expressing a receptor for a βγ subunit is incubated with labeled γ₂, γ₃, γ₄, γ₅, γ₇, γ₁₀ or γ₁₁ subunit in the presence of a test compound; and
(b) measuring the ability of this test compound to enhance the interaction or block the interaction of the receptor for a βγ subunit with a γ₂, γ₃, γ₄, γ₅, γ₇, γ₁₀ or γ₁₁ subunit.


SEQUENCE LISTING (23 sequences)

SEQ ID NO: 1 - protein, 74 aa, HOMO SAPIENS
MPVINIEDLTEKDKLKMEVDQLKKEVTLERMLVSKCCEEVRDYVEERSGEDPLVKGIPEDKNPFKELKGGCVIS

SEQ ID NO: 2 - protein, 71 aa, HOMO SAPIENS
MASNNTASIAQARKLVEQLKMEANIDRIKVSKAAADLMAYCEAHAKEDPLLTPVPASENPFREKKFFCAIL

SEQ ID NO: 3 - protein, 75 aa, HOMO SAPIENS
MKGETPVNSTMSIGQARKMVEQLKIEASLCRIKVSKAAADLMTYCDAHACEDPLITPVPTSENPFREKKFFCALL

SEQ ID NO: 4 - protein, 75 aa, HOMO SAPIENS
MKEGMSNNSTTSISQARKAVEQLKMEACMDRVKVSQAAADLLAYCEAHVREDPLIIPVPASENPFREKKFFCTIL

SEQ ID NO: 5 - protein, 69 aa, HOMO SAPIENS
  unsure (65): Deduced amino acid sequence of the cDNA clone for the
MSGSSSVAAMKKVVQQLRLEAGLNRVKVSQAAADLKQFCLQNAQHDPLLTGVSSSTNPFRPQKVXCSFL

SEQ ID NO: 6 - protein, 69 aa, HOMO SAPIENS
  unsure (65): Deduced amino acid sequence of the cDNA clone for the
MSATNNIAQARKLVEQLRIEAGIERIKVSKAASDLMSYCEQHARNDPLLVGVPASENPFKDKKPXCIIL

SEQ ID NO: 7 - protein, 69 aa, HOMO SAPIENS
  unsure (65): Deduced amino acid sequence of the cDNA clone for the
MSSGASASALQRLVEQLKLEAGVERIKVSQAAAELQQYCMQNACKDALLVGVPAGSNPFREPRSXCALL

SEQ ID NO: 8 - protein, 74 aa, HOMO SAPIENS
  unsure (70): Deduced amino acid sequence of the cDNA clone for the
MPALHIEDLPEKEKLKMEVEQLRKEVKLQRQQVSKCSEEIKNYIEERSGEDPLVKGIPEDKNPFKEKGSXCVIS

SEQ ID NO: 9 - DNA, 689 bp, HOMO SAPIENS
ggcacgagctcatctgacgactgacagctgatggcaccgccagcctctgtcccttggccaggactgtcacacggctgactctcagcaggggcagtagaatgaaagagggcatgtctaataacagcaccactagcatctcccaagccaggaaagctgtggagcagctaaagatggaagcctgtatggacagggtcaaggtctcccaggcagccgcggacctcctggcctactgtgaagctcacgtgcgggaagatcctctcatcattccagtgcctgcatcagaaaacccctttcgcgagaagaagttcttttgtaccattctctaactccgtgtgtgatgaaaacgcctccttttctgaccttcaaagtcccctgtagagaccatgcatgctctaagccttagggagtgagaccaacacccatccctgcccagccaacagtggccggggcttgtcttatgtttccatctgttttcttcgtggcattcaatttcatttttttccttttcattttcatgttattttcattattggcaaagaaaatcaaaatgtttatagccaaataacaaatgtgccatgtaaaagtaagtctggacttaagagtttaaaatttttaaacatcagtttccaagtttatatcatattaatacatttcagtggataatttatttaaaaaaaaaaaaaaaaaaa

SEQ ID NO: 10 - DNA, 1213 bp, HOMO SAPIENS
ggcacgagcccagcgccgccgccatgtcctccggggctagcgcgagcgccctgcagcgcttggtagagcagctcaagttggaggctggcgtggagaggatcaaggtctctcaggcagctgcagagcttcaacagtactgtatgcagaatgcctgcaaggatgccctgctggtgggtgttccagctggaagtaaccccttccgggagcctagatcctgtgctttactctgaagactctaggagagaagtttgctgaggaatgccttcaagcacaaagtgatgaatgactgccttcaagtctcaagaaaacacttttccctaacttttagagatatttcagccctttcctgtggcctggtcctatagccaaaatcacagatattcatgagtttctacttgagtgagaaaactgggtgaaggaatagaattttaaatagtaataactgcttgttttttgtgtgcaagtacttttatacataagataaacaaaaaccttaccaccaaacataccaaaatgcacctctttcataagtgagttactaagatttctatacctggaatatcatgtatgtttcatttactggatgtttacattttaggaaggaaaatagttttgtttatttaaacaactgaatacttataaactgttgttcctggaagttatttattccataaaaaatttgttcttttctcatgaatttataattcctaaatgaagaccagaaagtacaaattgctgggaggaagaataggctttattaatcaactgatgtcttgatttttctaaatgggaagattgctttatttttaacactaattatgggagcagattcttaccaaacttctttggaaaagttaatgttatgatgtgcattaggctgccccatcgtgtatataaatgaaggcagatttgatttttgtattcttacgtttactctgctttgtagttgtggctgtacttaaagcaatacagaatttcatatatttaaaaatgtttaaaatgtgacccacagaacattgtaaatgattaaaaactaacatgaaaatattacaacctaaaagaattcttaacttcacaagtgttttacttcgacgatgtgcctttgatttaatttgggacacttttttagaaggatacattattcgtgtttgcaacggtctttgaagagcttggaaataaaatttctgcttaattaaaaaaaaaaaaaaaaaaa

SEQ ID NO: 11 - DNA, 654 bp, HOMO SAPIENS
ggcacgagctcgtgccggccttcagttgtttcgggacgcgccgagcttcgccgctcttccagcggctccgctgccagagctagcccgagcccggttctggggcgaaaatgcctgcccttcacatcgaagatttgccagagaaggaaaaactgaaaatggaagttgagcagcttcgcaaagaagtgaagttgcagagacaacaagtgtctaaatgttctgaagaaataaagaactatattgaagaacgttctggagaggatcctctagtaaagggaattccagaagacaagaacccctttaaagaaaaaggcagctgtgttatttcataaataacttgggagaaactgcatcctaagtggaagaactagtttgttttagttttcccagataaaaccaacatgctttttaaggaaggaagaatgaaattaaaaggagactttcttaagcaccatatagatagggttatgtataaaagcatatgtgctactcatctttgctcactatgcagtcttttttaagagagcagagagtatcagatgtacaattatggaaataagaacattacttgagcatgacacttctttcagtatattgcttgatgcttcaaataaagttttgtcttaaaaaaaaaaaaaaaaaaaaaaaaaaaaaaaa

SEQ ID NO: 12 - DNA, 20 bp, HOMO SAPIENS
ctatccagcactccgatggc

SEQ ID NO: 13 - DNA, 20 bp, HOMO SAPIENS
ctatccagcactccgatggc

SEQ ID NO: 14 - DNA, 20 bp, HOMO SAPIENS
ctatccagcactccgatggc

SEQ ID NO: 15 - DNA, 20 bp, HOMO SAPIENS
gagctcagaggagagcacag

SEQ ID NO: 16 - DNA, 20 bp, HOMO SAPIENS
gtgcaccatgtctggctcct

SEQ ID NO: 17 - DNA, 20 bp, HOMO SAPIENS
cactggatcataaggagtgg

SEQ ID NO: 18 - DNA, 20 bp, HOMO SAPIENS
gatggcagacaatgtcagcc

SEQ ID NO: 19 - DNA, 20 bp, HOMO SAPIENS
agttataaaataatacaagg

SEQ ID NO: 20 - DNA, 827 bp, HOMO SAPIENS
ggcacgagcacatactcacaacgctgccgccgcgctccgtgggcaactcctactactgctgggctgggctgggctgggctgggctgcgccggagctcgcctgcacagatcagctccggagaggggaaaaccacgctcctcggaccaagcctcgggagctaagccagatctgccagtgagcctcaggctttaggaactgaagagtgtttctgaaagatctatccagcactccgatggccagcaacaacaccgccagcatagcacaagccaggaagctggtagagcagcttaagatggaagccaatatcgacaggataaaggtgtccaaggcagctgcagatttgatggcctactgtgaagcacatgccaaggaagaccccctcctgacccctgttccggcttcagaaaacccgtttagggagaagaagtttttctgtgccatcctttaagtctttgagaggggcctgaagagcctccgggctcctgggacattgatgtagagtttttagtgaagtgggcacctttctagtccacggcatttgaagagagcgaggagaaccattctggaaactctaggctatgcatgtttaaagatctggtcccctttatgagaatgcaagccgatccacatcctgacttaagagatctgattctgacgaactgcctggaggaggggaatatataaaaataaaattggtgtcacttcttttctgctatcccccagcccccccccccccaaaatcctcatgtttctgcttcatattttgaaaataacaattaaaacagacagctgttaaaaaaaaaaaaaaaaaaaaaaaaaaaaaaaaa

SEQ ID NO: 21 - DNA, 900 bp, HOMO SAPIENS
ggcacgagctgagaccagacctctggcctggccctccccaggggcctcctttcctatagtcactgcttctgcatcagatactttcagctgcaactccctactgggtggggcacccatttcaggcagaaggttttggtaccctccactgaccctacacccagggctgctactgccgcttgtggcttcaggatgaaaggtgagaccccggtgaacagcactatgagtattgggcaagcacgcaagatggtggaacagcttaagattgaagccagcttgtgtcggataaaggtgtccaaggcagcagcagacctgatgacttactgtgatgcccacgcctgtgaggatcccctcatcacccctgtgcccacttcggagaaccccttccgggagaagaagttcttctgtgctctcctctgagctcccctgtcccttctcacaactcctcccttttccctctcctgggcccttccttaggtcagtaattgttgtgagccccttaggctccttgcatcccatccctaacccttgcctgaccatgtgaggttatctgaagcacaaggcccaccctcacctatctgtcgaccccatttcctaccacctttgtggccgaccccaagcaccccagagatatgaggcaccctttgctccacccacagcagggccccgtcagactctgccagcgcgtcctgcccgcttccctcggtgacctgctcagacaatggagagggatgggccaggttcttgctctcagtctcacctggagctactgggagggtaaagccatttgaagaataaagtcatccagagcctcaaaaaaaaaaaaaaaaaaaaaaaaaaataaaaaaaaaaaaaaaaaaaaaaaaaaaaaaaaaaaaaaaaaaaaaaaaaaaaaaaaaaaaa

SEQ ID NO: 22 - DNA, 545 bp, HOMO SAPIENS
cccggggtctggccccgccgacccacggcccacgacccaccgacccacgaatcggcccggccgtcgcgtgcaccatgtctggctcctccagcgtcgccgctatgaagaaagtggttcaacagctccggctggaggccggactcaaccgcgtaaaagtttcccaggcagctgcagacttgaaacagttctgtctgcagaatgctcaacatgaccctctgctgactggagtatcttcaagtacaaatcccttcagaccccagaaagtctgttcctttttgtagtaaaatgaatctttcaaaggtttccccaaaccactccttatgatccagtggaatattcaagagagctacattttgaagcctgtacaaaagcttatccctgtaacacatgtgccataatatacaaacttctactttcgtcagtccttaacatctacctctctgaattttcatgaatttctatttcacaagggtaattgttttatatacactggcagcagcatacaataaaacttagccatgaaactttaaaaaaaaaaaaaaaaa

SEQ ID NO: 23 - DNA, 398 bp, HOMO SAPIENS
tgccgcggggctgaggcggccgcggggcccgagcgcagggagtggagcttggtttcgggatctcggtgctgcagacggcgagacctcctgcacagggtgtacagcaagctgtgattcctgggaaaactaaaaaagctctctggacaacggggcccagagctgatggcagacaatgtcagccactaacaacatagcccaggcccggaagctggtggaacagctacgcatagaagccgggattgagcgcatcaaggtctccaaagcggcgtctgacctcatgagctactgtgagcaacatgcccggaacgaccccctgctggtcggagtccctgcctcggagaacccctttaaggacaagaaaccttgtattattttataactgtgttctcactcgtgcc